(19) United States
(12) Patent Application Publication
(10) Pub. No.: US 2020/0303053 A1
(43) Pub. Date: Sep. 24, 2020

(54) NON-TRANSITORY STORAGE MEDIUM, INFORMATION PROCESSING METHOD, AND INFORMATION PROCESSING APPARATUS

(71) Applicant: TOYOTA JIDOSHA KABUSHIKI KAISHA, Toyota-shi (JP)

(72) Inventor: Takashige HORI, Aisai-shi (JP)

(73) Assignee: TOYOTA JIDOSHA KABUSHIKI KAISHA, Toyota-shi (JP)

(21) Appl. No.: 16/807,273

(22) Filed: Mar. 3, 2020

(30) Foreign Application Priority Data
Mar. 19, 2019 (JP) 2019-050933

(51) Int. Cl. G16H 20/30; G16H 20/60; G16H 10/60; G06Q 10/10; G01C 21/20

(52) U.S. Cl. CPC G16H 20/30; G16H 20/60; G01C 21/20; G06Q 10/109; G16H 10/60

(57) ABSTRACT

A non-transitory storage medium storing instructions that are executable by an information processing apparatus and that cause the information processing apparatus to execute a step of acquiring schedule information including a first schedule, a second schedule next to the first schedule, and an initial movement method from a first point related to the first schedule to a second point related to the second schedule, a step of correcting the initial movement method such that a consumption calorie required for a movement increases according to at least any one of a first free time from an end timepoint of the first schedule to a departure timepoint of the initial movement method and a second free time from an arrival timepoint of the initial movement method to a start timepoint of the second schedule to generate a corrected movement method, and a step of outputting the generated corrected movement method.

### INCORPORATION BY REFERENCE

The disclosure of Japanese Patent Application No. 2019-050933 filed on Mar. 19, 2019 including the specification, drawings and abstract is incorporated herein by reference in its entirety.

### BACKGROUND

### 1. Technical Field

The disclosure relates to a non-transitory storage medium, an information processing method, and an information processing apparatus.

### 2. Description of Related Art

Schedule management software for managing a schedule has been used. In such schedule management software, for example, schedules of various contents are registered together with dates and times and places related to the schedules. When one schedule ends, a user needs to move to a place related to a next schedule by a start timepoint of the next schedule.

In this regard, various navigation apparatuses for supporting the movement from a current location to a destination are used. In particular, recently, navigation apparatuses that take into account a consumption calorie of the user have been proposed for a purpose of contributing to health management of the user or the like. For example, Japanese Unexamined Patent Application Publication No. 2012-113516 (JP 2012-113516 A) discloses a navigation apparatus that predicts an amount of caloric value ingested by a user at a destination store, calculates a recommended amount of exercise and a recommended amount of consumed caloric value from the predicted amount of ingested caloric value, and searches for a route through which the recommended amount of consumed caloric value can be consumed by moving to the destination.

### SUMMARY

However, the navigation apparatus in the related art mostly focuses on calculating the shortest route or the fastest route from the current location to the destination. Therefore, a free time between one schedule and a next schedule is not considered.

The disclosure is to change a movement method in consideration of both a free time and user health management.

A non-transitory storage medium according to an aspect of the disclosure is a non-transitory storage medium storing instructions that are executable by an information processing apparatus and that cause the information processing apparatus to execute a step of acquiring schedule information including a first schedule, a second schedule next to the first schedule, and an initial movement method from a first point related to the first schedule to a second point related to the second schedule, a step of correcting the initial movement method such that a consumption calorie required for a movement increases according to at least any one of a first free time from an end timepoint of the first schedule to a departure timepoint of the initial movement method and a second free time from an arrival timepoint of the initial movement method to a start timepoint of the second schedule to generate a corrected movement method, and a step of outputting the generated corrected movement method. Accordingly, it is possible to change the movement method in consideration of both the free time and the user health management.

According to the disclosure, it is possible to change the movement method in consideration of both the free time and the user health management.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a diagram showing an example of a configuration of an information processing apparatus according to an embodiment;

FIG. 2 is a table showing an example of a data configuration of a schedule table;

FIG. 3 is a table showing an example of a data configuration of an ingestion calorie table;

FIG. 4 is a table showing an example of a data configuration of a consumption calorie table;

FIG. 5 is a diagram showing an example of an operation flow of the information processing apparatus;

FIG. 6 is a diagram showing an example of a schedule screen including an initial movement method; and

FIG. 7 is a diagram showing an example of a schedule screen including a corrected movement method.

### DETAILED DESCRIPTION OF EMBODIMENTS

Preferred embodiments of the disclosure will be described with reference to accompanying drawings. (In each drawing, elements to which the same reference numeral is assigned have the same or similar configuration.)

(1) Configuration

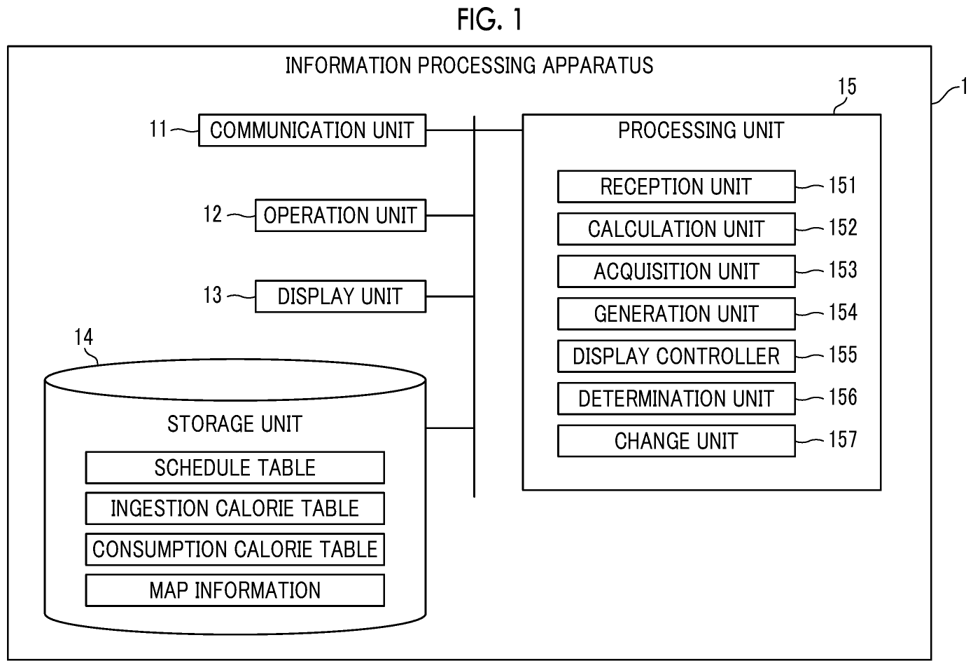

FIG. 1 is a diagram showing an example of a configuration of an information processing apparatus 1 according to the embodiment. The information processing apparatus 1 according to the embodiment is an information processing apparatus to manage a schedule of a user, for example. For this purpose, the information processing apparatus 1 includes, for example, a communication unit 11, an operation unit 12, a display unit 13, a storage unit 14, and a processing unit 15.

The communication unit 11 includes a communication interface circuit and connects the information processing apparatus 1 to a communication network. The communication unit 11 transmits data supplied from the processing unit 15 to another information processing apparatus or the like through the communication network. The communication unit 11 supplies data received from another information processing apparatus or the like to the processing unit 15 through the communication network.

The operation unit 12 may be any device as long as the information processing apparatus 1 can be operated, such as a touch panel or a key button. The user can input a character, a number, a symbol, or the like using the operation unit 12. When the user operates the operation unit 12, the operation unit 12 generates a signal corresponding to the operation. The generated signal is supplied to the processing unit 15 as a user instruction.

The display unit 13 may be any device as long as a video, an image, or the like can be displayed, such as a liquid crystal display or an organic electro-luminescence (EL) display. The display unit 13 displays a video corresponding to video data, an image corresponding to image data, or the like supplied from the processing unit 15.

The storage unit 14 includes, for example, a semiconductor memory apparatus. The storage unit 14 stores an operating system program, a driver program, an application program, data, and the like used for processing in the processing unit 15. For example, the storage unit 14 stores an input device driver program that controls the operation unit 12, an output device driver program that controls a store terminal display unit 34, and the like, as driver programs. In addition, the storage unit 14 stores a predetermined schedule management program and the like as the application program. The various programs may be installed in the storage unit 14 from a computer-readable portable recording medium such as a CD-ROM or DVD-ROM using a known setup program or the like. Further, the storage unit 14 may temporarily store temporary data related to predetermined processing. The storage unit 14 may store a schedule table, an ingestion calorie table, a consumption calorie table, map information, and the like. In the embodiment, the storage unit 14 is a non-transitory storage medium storing instructions that are executable by the information processing apparatus 1 and that cause the information processing apparatus 1 to execute the steps described below.

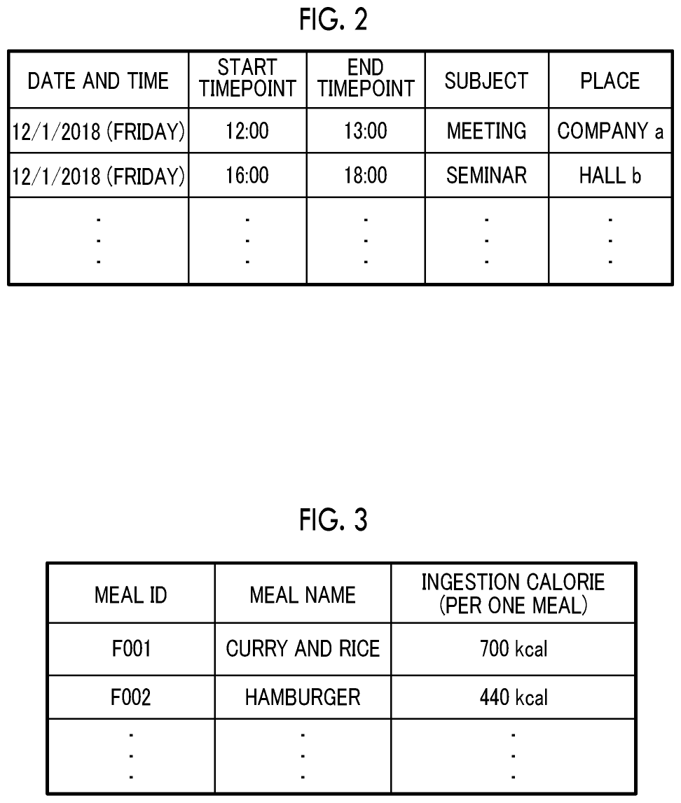

FIG. 2 is a table showing an example of a data configuration of the schedule table. The schedule table is a table for managing the schedule of the user. As shown in FIG. 2, the schedule table includes items such as “date and time”, “start timepoint”, “end timepoint”, “subject”, and “place”. Each item of the schedule table shown in FIG. 2 is an example, and the schedule table may have another item. The “date and time”, the “start timepoint”, and the “end timepoint” are information indicating a date and time, a start timepoint, and an end timepoint of the schedule, respectively. The “subject” is, for example, a name representing the schedule, which is randomly input by the user. The “place” is, for example, information indicating a place where the schedule is performed, which is randomly input by the user.

FIG. 3 is a table showing an example of a data configuration of the ingestion calorie table. The ingestion calorie table is a table that defines ingestion calories for each meal. As shown in FIG. 3, the ingestion calorie table may include, for example, “meal ID”, “meal name”, “ingestion calorie”, and the like. Each item in the ingestion calorie table shown in FIG. 3 is an example, and the ingestion calorie table may have another item or may be randomly set by the user or the like. The “meal” is identification information (ID) for identifying a meal. The “meal name” is information indicating a name or contents of the meal. The “ingestion calorie” is an amount of caloric value that can be ingested by taking the meal. A unit of the “ingestion calorie” may be represented as, for example, an amount of ingested caloric value per unit weight of a meal.

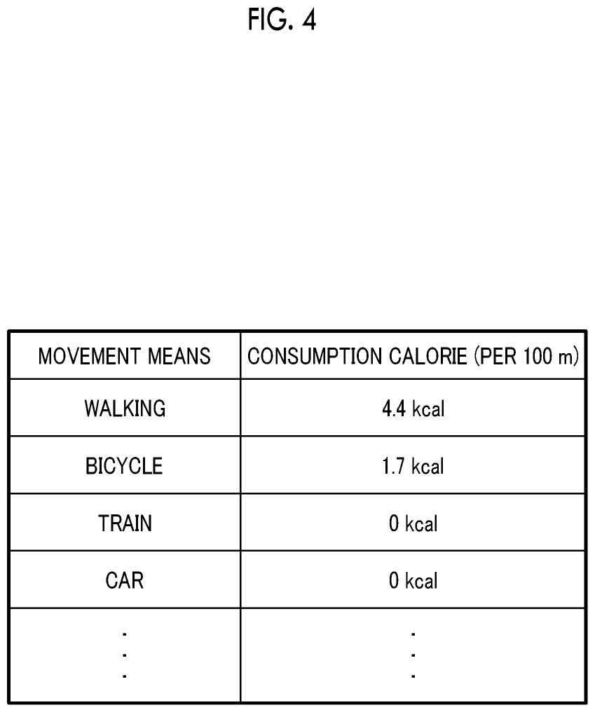

FIG. 4 is a table showing an example of a data configuration of the consumption calorie table. The consumption calorie table is a table that defines a consumption calorie per unit movement distance for each movement means. As shown in FIG. 4, the consumption calorie table may include, for example, “movement means”, “consumption calorie”, and the like. The “movement means” indicates various movement means. However, the “movement means” is not limited thereto and may include, for example, walking, bicycle, train, car, and the like. The “consumption calorie” is an amount of caloric value consumed when the user moves by the movement means. The unit of the “consumption calorie” may be represented as, for example, an amount of consumed caloric value per unit movement distance.

The processing unit 15 includes one or a plurality of processors and peripheral circuits thereof. The processing unit 15 integrally controls the overall operation of the information processing apparatus 1 and is, for example, a CPU. The processing unit 15 controls the operation of the communication unit 11, the display unit 13, or the like such that various types of processing of the information processing apparatus 1 are executed in an appropriate procedure based on the programs or the instructions stored in the storage unit 14, the operation of the operation unit 12, or the like. The processing unit 15 executes the processing based on the program (operating system program, driver program, application program, or the like) or the instructions stored in the storage unit 14. The processing unit 15 can execute a plurality of programs (application program and the like) or instructions in parallel. The processing unit 15 includes a reception unit 151, a calculation unit 152, an acquisition unit 153, a generation unit 154, a display controller 155, a determination unit 156, and a change unit 157.

(2) Operation

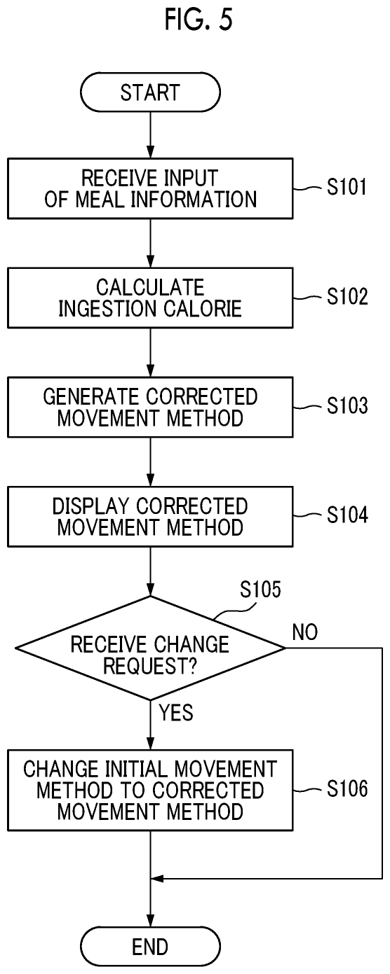

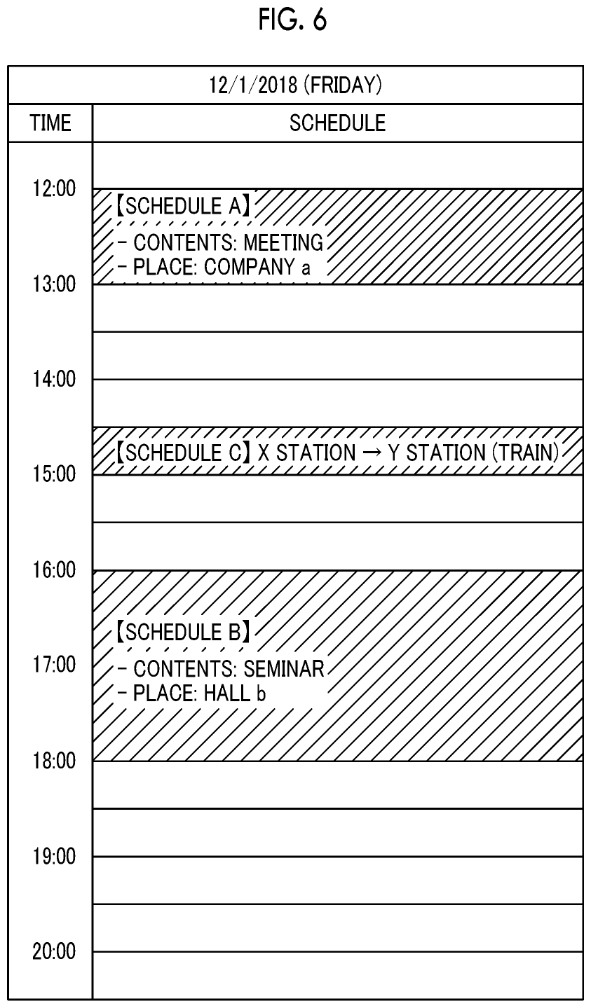

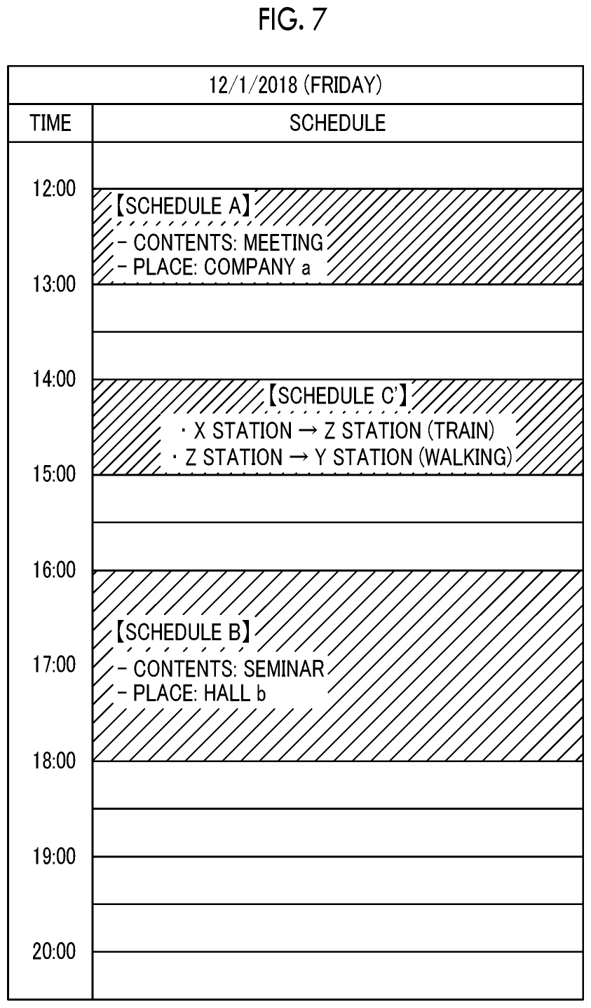

An example of an operation flow of the information processing apparatus 1 according to the embodiment will be described with reference to FIGS. 5, 6, and 7. FIG. 5 is a diagram showing an example of the operation flow of the information processing apparatus 1. FIG. 6 is a diagram showing an example of a schedule screen including an initial movement method. FIG. 7 is a diagram showing an example of a schedule screen including a corrected movement method.

First, the schedule shown in FIG. 2 is assumed to be registered in the schedule table as a premise of the following operation flow. That is, a schedule A, a schedule B after the schedule A, and a movement C between the schedule A and the schedule B are registered on Dec. 1, 2018 (Friday) as shown in FIG. 2. The schedule A is a meeting held at a company a starting at 12:00 and ending at 13:00. The schedule B is a seminar held at Hall b starting at 16:00 and ending at 18:00. The movement C is a movement by train that departs from X station at 14:30 and arrives at Y station at 15:00. Here, the movement C is an example of the initial movement method and may be, for example, the fastest route or the shortest route from the company a to the Hall b.

First, the reception unit 151 receives an input of meal information is received in response to an operation of the operation unit 12 by the user (S101). Next, the calculation unit 152 refers to the ingestion calorie table based on the received input meal information to calculate an ingestion calorie from the meal (S102).

Next, the generation unit 154 generates a corrected movement method by correcting the initial movement method (S103). Here, the initial movement method is corrected such that the consumption calorie required for the movement increases. An amount of increase in consumption calorie may be calculated based on the ingestion calorie calculated in S102. In the correction of the initial movement method, for example, movement means (initial movement means) related to the initial movement method may be corrected, or a movement route (initial movement route) related to the initial movement method may be corrected. When the initial movement means is corrected, for example, the initial movement means (train in this example) may be corrected to movement means with high consumption calorie (for example, bicycle or walking) with reference to the consumption calorie table. In addition, when the initial movement route is corrected, the initial movement route may be corrected to a movement route having a longer distance or a longer required time with reference to the map information. As described above, the corrected movement method is generated by correcting the initial movement method.

Next, the display controller 155 displays, for example, the schedule screen shown in FIG. 7 to display the corrected movement method (S104). In the example shown in FIG. 7, a movement C′ is shown as the corrected movement method. Next, the determination unit 156 determines whether or not a change request of the movement method from the user is received (S105). When determination is made that the change request of the movement method is received, the initial movement method is changed to the corrected movement method in the schedule table (S106) and the processing ends. Alternatively, when determination is made that the change request of the movement method is not received, the processing ends.

As described above, the information processing apparatus according to the embodiment acquires schedule information including a first schedule, a second schedule next to the first schedule, and an initial movement method from a first point related to the first schedule to a second point related to the second schedule, corrects the initial movement method such that a consumption calorie required for the movement increases according to a free time from an arrival timepoint of the initial movement method to a start timepoint of the second schedule to generate a corrected movement method, and outputs the generated corrected movement method. Accordingly, the change of the movement method in consideration of both the free time and the user health management is achieved.

The embodiments described above are intended to facilitate the understanding of the disclosure and are not intended to limit the disclosure. Each element included in the embodiments and the disposition, material, condition, shape, size, and the like thereof are not limited to the above examples and can be changed as appropriate. The configurations shown in the different embodiments can be partially replaced or combined with each other.

What is claimed is:

1. A non-transitory storage medium storing instructions that are executable by an information processing apparatus and that cause the information processing apparatus to execute steps of:
acquiring schedule information including a first schedule, a second schedule next to the first schedule, and an initial movement method from a first point related to the first schedule to a second point related to the second schedule;
correcting the initial movement method such that a consumption calorie required for a movement increases according to at least any one of a first free time from an end timepoint of the first schedule to a departure timepoint of the initial movement method and a second free time from an arrival timepoint of the initial movement method to a start timepoint of the second schedule to generate a corrected movement method; and
outputting the generated corrected movement method.

2. The non-transitory storage medium according to claim 1, wherein the correcting the initial movement method such that a consumption calorie required for a movement increases includes at least one of changing initial movement means related to the initial movement method to corrected movement means having a larger consumption calorie per unit movement than the initial movement means and changing an initial movement route related to the initial movement method to a corrected movement route longer than the initial movement route.

3. The non-transitory storage medium according to claim 1, wherein the step of generating the corrected movement method includes advancing the departure timepoint of the initial movement method by a predetermined time according to the first free time from the end timepoint of the first schedule to the departure timepoint of the initial movement method.

4. The non-transitory storage medium according to claim 1, wherein the non-transitory storage medium further stores an instruction that causes the information processing apparatus to execute a step of:
receiving an input of an ingestion calorie by a user,
wherein the step of generating the corrected movement method is executed based on the ingestion calorie.

5. An information processing method causing an information processing apparatus to execute the steps of:
acquiring schedule information including a first schedule, a second schedule next to the first schedule, and an initial movement method from a first point related to the first schedule to a second point related to the second schedule;
correcting the initial movement method such that a consumption calorie required for a movement increases according to at least any one of a first free time from an end timepoint of the first schedule to a departure timepoint of the initial movement method and a second free time from an arrival timepoint of the initial movement method to a start timepoint of the second schedule to generate a corrected movement method; and
outputting the generated corrected movement method.

6. An information processing apparatus comprising:
an acquisition unit that acquires schedule information including a first schedule, a second schedule next to the first schedule, and an initial movement method from a first point related to the first schedule to a second point related to the second schedule;
a generation unit that corrects the initial movement method such that a consumption calorie required for a movement increases according to at least any one of a first free time from an end timepoint of the first schedule to a departure timepoint of the initial movement method and a second free time from an arrival timepoint of the initial movement method to a start timepoint of the second schedule to generate a corrected movement method; and
an output unit that outputs the generated corrected movement method.
